(19) United States
(12) Patent Application Publication
(10) Pub. No.: US 2019/0183792 A1
(43) Pub. Date: Jun. 20, 2019

(54) COMPOSITION AND METHOD FOR TREATING HERPESVIRUS IN MAMMALS

(71) Applicant: VELOCE BIOPHARMA, LLC, Fort Lauderdale, FL (US)

(72) Inventors: Joseph CAPRIOTTI, Christiansted, VI (US); Kara CAPRIOTTI, Fort Washington, PA (US); Jesse PELLETIER, Miami, FL (US); Kevin STEWART, Christiansted, VI (US)

(21) Appl. No.: 16/327,632

(22) PCT Filed: Aug. 22, 2017
(86) PCT No.: PCT/US17/48027

§ 371 Date: Feb. 22, 2019

Related U.S. Application Data
(60) Provisional application No. 62/378,050, filed on Aug. 22, 2016.

(51) Int. Cl. A61K 9/00; A61K 31/79; A61K 47/20; A61K 9/06; A61K 47/38; A61P 31/22

(52) U.S. Cl. CPC A61K 9/0048; A61K 31/79; A61P 31/22; A61K 9/06; A61K 47/38; A61K 47/20

(57) ABSTRACT

Disclosed is a composition and method for ocular infection, including treating ocular herpesvirus infection, including feline herpesvirus (FHV), canine herpesvirus (CHV), and equine herpesvirus (EHV), where the composition includes povidone-iodine and DMSO.

### BACKGROUND OF THE INVENTION

The invention relates to methods of treatment involving stable topical formulations useful in the treatment of viral infection of the ocular surface and periocular area in non-human mammals. The invention also relates to methods for the treatment of dry eye disease and ocular surface dysfunction in non-human mammals.

Feline herpesvirus (FHV), and particularly FHV type-1 (FHV-1), is a highly contagious virus that is one of the major causes of upper respiratory infections (URIs) or cat flu in cats. In addition, FHV has been described as the most common viral cause of ocular surface disease, such as feline herpes keratitis, in cats. The feline herpesvirus I (FHV-1) is the causative virus of Feline Viral Rhinotracheitis (FVR), an upper respiratory tract infection, and associated viral eye infections. It has the ability to persist in latent form in the eye making reoccurrence (or recrudescence) of infection common. Eighty percent of cats become carriers following primary infection and vaccinated cats may become chronic carriers without evidence of clinical disease. Recrudescence may be associated with stress, surgery, lactation, corticosteroid therapy, and immunosupression from Feline Leukemia Virus (FeLV) or Feline Immunodeficiency Virus (FIV) infection.

FHV-1 propagates readily in the epithelium of the respiratory tract and the conjunctiva. The course of infection in the conjunctival epithelium is self-limiting. Epithelial damage is evident in two days and is most pronounced in seven to ten days, post infection. Intranuclear inclusion bodies are readily identified in histologic section of affected tissue, but they are less readily identified on cytological examination of conjunctival scrapings.

Acute FHV-1 infection usually occurs in neonatal and young adult cats. The upper respiratory signs predominate with sneezing and nasal and ocular discharge being most notable. The ocular signs include bilateral conjunctivitis, serous ocular discharge which may become mucoid or mucopurulent, and blepharospasm. Corneal lesions are much less conspicuous during acute primary FHV-1 infection. Microdendritic lesions may be seen between three and six days after infection. Larger dendrites may be seen around day eleven after a new wave of virus particles are released from degenerating conjunctiva. These lesions are subtle and only occasionally do the dendrites coalesce to form larger geographic lesions. Corneal infection at this stage results in relatively little cytopathic effect, with no inflammatory response. A mild transient superficial vascularization may occur.

It is during this stage that viral latency is established. It is reactivation of latent virus that is responsible for the recrudescent infection seen in older cats. Recrudescent infections generally occur in adult cats that presumably have recovered from primary FHV-1 infection earlier in life. In the adult cat, recrudescent FHV-1 infections may cause conjunctivitis only, or may also cause keratitis. Upper respiratory tract symptoms are usually absent. The conjunctival form generally consists of mild conjunctival hyperemia, and intermittent ocular discharge. The course may be prolonged and often is recurrent.

Should corneal involvement occur, dendritic epithelial lesions may be seen using a vital stain such as Rose Bengal. The dendrites often coalesce to form geographic “map like” lesions. Often mild stromal edema and superficial stromal vascularization follow. Stromal involvement in herpetic keratitis is of great concern due to the marked amount of stromal scarring that can occur. This form is characterized by stromal edema, deep vascularization, inflammatory cell infiltrates and ultimately collagen damage and opacification. It is thought that the stromal form of FHV-1 keratitis is not a direct effect of the virus on keratocytes, but a pathologic immune reaction to viral antigen which is mediated by cytotoxic T lymphocytes.

Complications of herpetic keratitis include the development of keratoconjunctivitis sicca (dry eye), symblepharon (conjunctival to corneal adhesions) and corneal sequestration. KCS may occur as a result of lacrimal adenitis or it may be due to ductile obstruction in cats with FHV-1 conjunctivitis. The sicca tends resolve when the inflammation subsides. Symblepharon may occur wherever there is significant damage to the conjunctival epithelium, and as such may occur follow FHV-1 conjunctivitis. Feline sequestrum may occur whenever there is chronic damage or irritation of the corneal stroma, and may follow FHV-1 keratitis.

The diagnosis of FHV-1 ocular infections is based upon the clinical history, the findings on clinical examination and the results of the appropriate diagnostic tests. Corneal dendrites may be very subtle, and it is necessary to use Rose Bengal, a vital stain to detect them. Since full thickness epithelial erosions generally occur only in advanced disease, fluorescein stain may not be helpful. Virus isolation, which for many years was the most sensitive means of making a positive diagnosis of feline herpesvirus ocular disease, has for the most part been replaced with the Polymerase Chain Reaction (PCR) test. A commercial virus collection swab is moistened with transport medium and rolled in the conjunctival fornix. The swab is then placed into the transport medium and submitted to the lab.

Conjunctival cytology is not a reliable method of diagnosing FHV-1 ocular disease. Intranuclear inclusion bodies are readily seen in histologic section of affected tissue, they are often not apparent on conjunctival smears. The presence of intracytoplasmic inclusion bodies characteristic of chlamydial infection is more apparent with Giemsa staining of conjunctival smears. Conjunctival cytology is therefore of value in ruling out chlamydial conjunctivitis. It is unusual for chlamydia, mycoplasma or calicivirus ocular infections to cause corneal lesions.

The treatment of acute FHV-1 infection in young cats when upper respiratory involvement is present includes broad spectrum antibiotics given systemically and applied topically. General support may be required in compromised neonates and this may include fluids, heat and the use of a vaporizer. If corneal involvement is present, topical antivirals are prescribed. In the adult cat with recrudescent FHV-1 infection topical antivirals are best used aggressively early in the course of the disease before stromal involvement progresses.

In an ocular disease process similar to that of Feline Herpes virus-1 (FHV-1), Canine Herpes Virus-1 or CHV-1 can infect the ocular mucosa, conjunctiva and cornea of canines. CHV-1 is classified as an alphaherpesvirus with many of the characteristics of Herpesviridae including short replication cycles, induction of lifelong latency, and narrow host range. CHV-1 is of particular concern in puppies, where the virus can cause a fatal hemorrhagic infection leading to focal organ necrosis and death. In the adult dog, the infection is usually asymptomatic but can manifest as upper respiratory tract infection and ocular disease.

Ocular infection in canines by CHV-1 has only recently been described. CHV-1 can present in the mature dog in a variety of ways affecting both the ocular adenexa and ocular surface. These manifestations include blepharitis, conjunctivitis, ulcerative keratitis and non-ulcerative keratitis. These conditions can develop during primary and/or recurrent infection with CHV-1. Because of the relationship to other Herpesviridae it is thought that its natural history and epidemiology are similar to FHV-1.

In an ocular disease process similar to FHV/CHV, Equine Herpes virus-1 and 2 have been implicated in causing ocular disease in horses. There are multiple serotypes of Equine Herpes virus and most are responsible for morbidity outside of the eye. EHV-2 is classified as a gamma herpes virus, which distinguishes it from others in the family Herpesviridae because of reproduction at a more variable rate than other subfamily members. EHV-2 has been implicated in keratoconjunctivitis including the epidemic type, conjunctivitis and keratitis. EHV-1 has been implicated in multifocal chorioretinitis, which has been postulated to occur via vascular injury to the eye after viremia. Because of the relationship to other Herpesviridae it is thought that natural history and epidemiology of EHV-1 are similar to FHV/CHV. With respect to EHV-2 and the gammaviridae, patterns and rates of replication are likely more akin to other members of this subfamily.

EHV-2 and the Gammaviridae are a subfamily of viruses in the family Herpesviridae. There are currently 32 species among the latter subfamily with mammals serving as natural hosts. These viruses are most notable for causing certain infections among humans via the Epstein-Barr virus or Kaposi's sarcoma associated herpes virus. Accordingly, the Gammaviridae may replicate and persist in lymphoid, fibroblast, or epithelial cells. Equine herpes virus-2 or EHV-2 is further classified in the genus Rhadinovirus where it is known for slow replication in equine tissues including the ocular mucosa and lymph nodes. Its replicative cycle is similar to other herpes viruses in that it is nuclear and lysogenic. Rhadinoviruses are also unique because of their ability to pirate cellular genes from their host cells and incorporate into their genome. The cyclin gene is one such example that regulates cell division and may be responsible for oncogenic phenomenon.

Three antiviral medications are available. Idoxuridine (IDU) interferes with viral DNA synthesis by substituting for the essential nucleotide thymidine. IDU is poorly soluble and is virostatic. Prolonged contact with the infected tissue is required. The 0.1% solution must be applied five times daily. IDU is marketed as Stoxil (Smith Kline & French) and Herplex (Allergan Pharmaceuticals). Adenine Arabinoside inhibits viral DNA polymerase and thus decreases viral DNA synthesis. It is sold as Vira A (Parke Davis) and the 3% ointment is applied five times daily. Triflurothymidine is incorporated into the viral DNA and leads to the synthesis of defective proteins. Sold as Viroptic (Burroughs Wellcome) it is considered the most effective, least toxic and most soluble of the antivirals. It is also the most expensive. Available as a 1% solution, Viroptic is applied every two hours while the animal is awake until the cornea has re-epithelialized, then reduced to every four hours while awake, for two weeks more. Acyclovir is the newest antiviral available for human herpesvirus. It is a tablet given orally and is marketed under the trade name Zovirax (Burroughs Wellcome). Research by Nasisse indicates that triflurothymidine is the most effective against Feline Herpesvirus followed by Idoxuridine then Adenine Arabinoside. Although Acyclovir was most effective against Human herpes simplex virus, it was not effective against Feline Herpesvirus. This may be due to the inability of FHV-1 to induce deoxycytidine kinase.

Other strategies to assist in the treatment of herpetic keratitis reported in the last few years include topically applied interferon, oral administration of 1-lysine and oral administration of cimetidine. Interferon is reported to stimulate the local immunity to viral infection. A 20 to 50 IU/ml solution in artificial tear is administered topically twice daily. Orally administered L-lysine has been reported to inhibit herpesvirus growth by competitive inhibition of the uptake of arginine which is required for viral multiplication. It is given to cats at a dosage of 125 mg twice daily. Cimetidine has been shown to generally stimulate cell mediated immunity. it is given at a dosage of 50 mg once daily. Anecdotal reports suggest that maintenance of cats on one or more of these medications during periods of quiescence, will result in a longer period of time before recrudescence of infection occurs, and that when recrudescence occurs, the infection in milder and of shorter duration.

Corticosteroids may be used in the treatment of chronic herpetic stromal keratitis to suppress the potentially scarring immune response if used carefully and in conjunction with an antiviral agent. Steroids are contraindicated if epithelial or conjunctival involvement is still active, because they delay re-epithelialization, prolong virus shedding and may allow conjunctival and corneal epithelial infection to involve the corneal stroma. Alternatively, topical Cyclosporine may be used with caution to reduce the scarring associated with herpesvirus stromal keratitis.

Certain antiviral drugs are currently used to treat ocular FHV, CHV, or EHV infection, but are disadvantageous because they are generally costly drugs, require frequent topical application or have undesirable side effects. In addition, most current therapies are not supported by well-controlled in vivo studies that justify their clinical use. For example, the anti-herpes nucleoside analogues, cidofovir and acyclovir, which are commonly used clinically to treat ocular herpesvirus infection in cats, have previously been evaluated in traditional two-dimensional feline cell cultures in vitro. Cidofovir has also been evaluated in canine models in vivo and was shown to decrease viral shedding, but local ocular toxicity is significant.

Therefore, novel treatment options for treating ocular FHV, CHV, or EHV infections that are efficacious, cost-effective, and require less frequent administration, are needed.

### SUMMARY OF THE INVENTION

The present invention employs a combination of penetration enhancer and antiseptic ingredient in order to deliver an effective therapeutic dose of a medicament, including by way of non-limiting example members of the antiseptic class of iodophor agents, with dimethyl sulfoxide employed as penetration enhancers. The compositions are employed to treat a variety of non-human mammalian infections of the periocular skin and ocular surface. Such a strategy is ideal because it allows polyantimicrobial agents to quickly and efficiently be delivered to and through the skin of the periocular region and through the cornea, conjunctiva and other ocular surface structures in non-human mammals in an effective, non-toxic concentration via a safe and convenient route.

In an embodiment, disclosed herein is a composition for treating ocular feline herpesvirus (FHV) infection, including FHV type 1 (FHV-1). As used herein, the terms “feline herpesvirus” and “FHV” are used interchangeably, and encompass, all types of feline herpesvirus, including feline herpesvirus type 1 (FHV-1). A composition of the subject invention comprises polyvinylpyrrolidone-iodine (PVP-I) and dimethylsulfoxide (DMSO), wherein the composition is effective to treat FHV infection without the use of costly nucleoside analogues antiviral drugs.

In an embodiment, a composition disclosed herein for treatment of an FHV infection is substantially anhydrous. In an embodiment, the composition is anhydrous.

In an embodiment, a composition disclosed herein for treatment of an FHV infection includes PVP-I at 0.01% to less than 0.5% (w/w). In an embodiment, PVP-I is present in a range selected from the group consisting of about 0.05% to about 0.4%, about 0.1% to about 0.3%, about 0.2% to about 0.275%. In an embodiment, PVP-I is present in an amount selected from the group consisting of about 0.1%, about 0.15%, about 0.2%, about 0.25%, about 0.3%, about 0.35%, about 0.4%, about 0.45%. Preferably, a composition of the subject invention comprises about 0.25% PVP-I.

In an embodiment, a composition disclosed herein for treatment of an FHV infection further comprises at least one antifungal agent selected from the group consisting of tolnaftate, terbinafine, undecylenic acid, clioquinol, miconazole, miconazole nitrate, clorrinazole, tioconazole, nystatin, terconazoic, butoconazole nitrate, ciclopirox olamine, econazole nitrate, triacetin, flucytosine, haloprogin, and ketoconazole. In an embodiment, an antifungal agent is present in an amount of about 1% to about 25% (w/w).

In an embodiment, disclosed herein is a method for treating an FHV, CHV, or EHV infection, comprising contacting an infected corneal surface with a composition disclosed herein, and repeating the contacting step as necessary until the FHV, CHV or EHV infection has been treated. In an embodiment, the contacting step is conducted at least once a day. In an embodiment, the contacting step is conducted at least twice a day.

In an embodiment, disclosed herein is a method for treating an FHV, CHV, or EHV infection, comprising contacting an infected corneal surface with a composition disclosed herein and repeating the contacting step for a period of about three days to about two weeks. In an embodiment, the contacting step is repeated for about one week.

### DETAILED DESCRIPTION

The present invention is directed towards methods of ocular surface and periocular skin topical treatment in non-human mammals with dimethyl sulfoxide (DMSO)/povidone-iodine (PVP-I) containing compositions. The invention is surprisingly useful for the treatment of viral, fungal and bacterial infections.

A specific but non-limiting example of a formulation that leads to a useful pharmaceutical preparation which can be employed in this method consists of solid PVP-I dissolved or suspended in DMSO. This formulation can be used to treat feline herpetic keratitis and feline conjunctivitis.

In an embodiment, a composition comprises an iodophor in the range of about 0.01% to about 15%. In another embodiment, a composition comprises an iodophor in the range between 0.05% and 12.5%. In another embodiment, a composition comprises an iodophor in the range between 0.05% and 10.0%. In another embodiment, a composition comprises an iodophor in the range between 0.1% and 10.0%. In another embodiment, a composition comprises an iodophor in the range between 0.1% and 5.0%. In another embodiment, a composition comprises an iodophor in the range between 0.25% and 9.0%. In another embodiment, a composition comprises an iodophor in the range between 0.2% and 2.5%.

In another embodiment, a composition comprises an iodophor in the range between 0.5% and 7.5%. %. In another embodiment, a composition comprises an iodophor in the range between 0.5% and 1.0%. In another embodiment, a composition comprises an iodophor in the range between 0.75% and 5.0%, and in yet another embodiment, between 1.0% and 4.0%. In an embodiment, a composition comprises an iodophor in the range of about 0,1% to about 2.5%. about 0.2% to about 2.0%. about 0.3% to about 1.0%. and about 0.4% to about 0.75%

In another embodiment, DMSO can be added to aqueous solutions of PVP-I. In an example DMSO can be present as a co-solvent with water in the range of 10%-99%. One embodiment of such a formulation could include a range of excipients such as sodium chloride, sodium dihydrogen phosphate monohydrate, disodium hydrogen phosphate anhydrous and water, as well as others known to those skilled in the art.

Povidone-iodine (PVP-I) is a vs own antiseptic to almost every medical specialty. It has gained recent interest beyond simple disinfection as a therapy for active infections in the eye, ear, sinuses, articular compartments and skin. A variety of organic solvents are known to enhance the percutaneous absorption of medicaments, including dimethylsulfoxide (DMSO). However, prior to the inventors' own discovery, DMSO was not known to be useful as a penetration enhancer with large (high molecular weight) polymeric compounds such as PVP-I. Accordingly, use of DMSO as a solvent in compositions comprising PVP-I was effectively avoided for therapeutic preparations.

In an aspect, as further described herein, it was surprisingly found that compositions comprising comparatively low concentrations (less than 0.5% w/w) PVP-I and DMSO at concentrations greater than 30%, preferably 30%-75%, more preferably 30% to 45%, and most preferably about 44%, are effective for treating FHV infections of the corneal surface. This is advantageous and surprising, in part, because PVP-I is generally known to be an irritant when applied directly to the corneal surface and can cause inflammation, redness and burning sensation. A lower dose of PVP-I, such as less than 5%, and preferably about 0.25% PVP-I can be highly efficacious, without causing irritation, redness, or burning.

In another aspect, as further described herein, is was surprisingly found that the compositions and methods encompassed herein are useful for treating ocular conditions in addition to FHV infections, including, but not limited to, fungal infections, yeast infections, other viral infections, and bacterial infections, including bacterial infections caused by Gram-positive and Gram-negative bacteria in the eye. A composition in accordance with the subject invention can therefore also include at least one additional antimicrobial agent (in addition to PVP-I). For example, a composition of the subject invention can comprise at least one additional antifungal agent for treating fungal infection, at least one additional anti-yeast agent for treating yeast infection, at least one additional antiviral agent for treating other viral infection, or at least one additional antibacterial agent for treating antibacterial infection.

Contemplated herein are compositions comprising at least one solvent which is a penetrant and at least 0.1%, preferably about 0.25% PVP-I for the treatment of ocular FHV infection in an animal. An animal can preferably be a feline, e.g., a domestic cat, but can include animals in the family of canine, equine, porcine, and the like, and methods of using a composition of the invention to treat ocular herpesvirus infection in the animal in need of treatment.

In an embodiment, disclosed herein are compositions comprising >30% DMSO and less than 0.5% PVP-I. A composition of the invention preferably comprises PVP-I which is completely dissolved in the solvent. In one preferred embodiment, no solid or undissolved PVP-I is present as a particulate or suspension in the composition.

Also disclosed herein are methods of using the described PVP-I/DMSO compositions. A composition of the subject invention can comprise a co-solvent. A co-solvent can be water, or an aqueous co-solvent, or can be an anhydrous alcohol or anhydrous organic co-solvent. The co-solvent can be a glycol or polyglycol. The composition can be free of glycol or can be free of polyglycol.

In addition, the composition can comprise a gelling agent which can provide viscous properties to the composition such that the composition is a gel, salve, cream, or ointment which is stable for purposes of efficacy, acceptable residence time at the site of application or use, and storage properties, e.g., having a pharmaceutically acceptable shelf-life. The comparatively higher viscosity composition comprising a gelling agent, as compared to a liquid composition, having comparatively lower viscosity, where no gelling agent is present, can be advantageous for localizing the composition on the ocular surface for longer periods of time. For example, a liquid composition applied to the ocular surface can be washed away by tearing in less than one hour, typically in less than one-half hour, and commonly within 10-15 minutes of initial administration. A gel composition can remain on the ocular surface for more than 15 minutes, commonly more than one-half hour, and preferably up to at least one hour and greater than one hour.

An ophthalmic gel composition of the invention can include a gelling agent, as is well known in the art, selected from a gum, agar, carrageenan, petrolatum, or a cellulosic polymer or the like. One preferred cellulosic polymer useful as a gelling agent is hydroxyethyl cellulose (HEC). An alternative cellulosic polymer gelling agent is hydroxymethyl cellulose (HMC) or hydroxypropyl methylcellulose (HPMC).

A composition of the invention preferably comprises povidone-iodine, or PVP-I having an average molecular weight greater than 10,000. More preferably, the composition of the invention comprises PVP-I having an average molecular weight between about 20,000 to about 1,000,000. One preferred embodiment comprises PVP-I having an average molecular weight between about 30,000 to about 60,000, or greater. Each of the PVP-I ingredients is referred to herein as a “high molecular weight PVP-I,” or “HMW PVP-I.”

Another embodiment of a composition according to the subject invention is a stable, topical ophthalmic gel formulation comprising about 0.1% to about 10% povidone-iodine (PVP-I); about 30% to about 99% dimethyl sulfoxide (DMSO); and about 1% to about 10% gelling agent; wherein, each ingredient in the composition is ophthalmically acceptable, and the ophthalmic gel composition exhibits greater efficacy in treating infectious conditions of the eye or eyelid, compared to a liquid composition substantially free of a gelling agent and comprising about 0.1% to about 10% povidone-iodine and about 30% to about 99% DMSO.

A preferred ophthalmic gel composition comprises about 0.5% to about 5% PVP-I. A more preferred ophthalmic gel composition comprises about 1% to about 3% PVP-I, and a most preferred ophthalmic gel composition comprises about 1% PVP-I.

A composition of the invention preferably comprises povidone-iodine, or PVP-I having an average molecular weight greater than 10,000. More preferably, the composition of the invention comprises PVP-I having an average molecular weight between about 20,000 to about 1,000,000. One preferred embodiment comprises high molecular weight (HMW) PVP-I.

A preferred ophthalmic gel composition comprises about 30% to about 70% DMSO. A more preferred ophthalmic gel composition can comprise about 40% to about 49% DMSO, and even more preferably, about 44% DMSO.

A preferred ophthalmic gel composition comprises about 0.25% to about 5% gelling agent. A more preferred ophthalmic gel composition can comprise about 0.5%-3.5% % gelling agent. A particularly useful composition which has been prepared for use in treating infection of the eye or eyelid comprises 1% PVP-I; 44% DMSO; and hydroxyethylcellulose (HEC); and the balance aqueous solvent and other inactive excipients. A preferred aqueous solvent is water or isotonic buffer.

### Therapeutic Indications

In an embodiment, disclosed herein are compositions and methods for treating FHV infection on the ocular surface. Therefore, the compositions and methods are useful for treatment of the eye, or eye surfaces, such as the cornea, and areas adjacent to or contacting the ocular surface, such as the eyelid. The compositions and methods are further useful in treating any combination of the above.

The term “treating”, as used herein, refers to a detectable improvement in an adverse condition and/or a lessening the symptoms of the condition upon contacting an ocular surface in a mammal with a composition disclosed or encompassed by the disclosure herein, The term “treating” encompasses both a partial improvement in an adverse condition and a complete eradication (i.e., “cure”) of the condition. In an aspect, an infection is treated.

The compositions and methods are useful for treatment of infections involving, but not limited to, viral infection, including feline herpesvirus (FHV).

The compositions and methods are useful in treating one or any combination of at least two of the above diseases, conditions or pathogens.

### Compositions

In an embodiment, a composition comprises at least one therapeutic agent and at least one solvent and/or penetrant. In an aspect, an iodophor is a therapeutic agent. In an embodiment, a composition comprises at least one antiseptic compound. In an aspect, an antiseptic compound is a therapeutic agent. In an embodiment, a composition comprises an iodophor antiseptic. “Iodophor”, as used herein, refers to a substance comprising iodine and at least one additional agent (e.g., a solubilizing agent) that releases. free iodine when in solution. Examples of iodophors include, but are not limited to, povidone iodine (PVP-I), iodine tincture, Lugol's solutions, and iodine salts (e.g., potassium iodide, sodium iodide).

In an embodiment, a composition comprises at least one iodophor. The compositions encompass any iodophor, as well as iodophors as yet to be developed or discovered. In an embodiment, the iodophor is PVP-I. In another embodiment, a composition comprises iodine. In an embodiment, a composition comprises iodine and at least one iodophor. In an embodiment, the composition comprises PVP-I and DMSO and is free of additional or supplemental iodophors and elemental iodine.

In an embodiment, PVP-I functions an active ingredient or a therapeutic agent in a composition. In an aspect, a PVP-I therapeutic agent functions as an antiseptic. In another embodiment, PVP-I functions as a preservative in a composition. In another aspect, a PVP-I preservative functions as a stabilizer. In an embodiment, PVP-I functions in at least once capacity in a composition. In another embodiment, PVP-I functions in at least two capacities in a composition. In another embodiment, a composition of the invention can be free of additional preservative or stabilizer.

In another embodiment, a composition further comprises at least one non-iodophor, non-iodine therapeutic agent. In an embodiment, the at least one non-iodophor, non-iodine therapeutic agent is an antiseptic. In another embodiment, the at least one non-iodophor, non-iodine therapeutic agent is not an antiseptic. In an embodiment, the composition of the invention can comprise PVP-I and DMSO and be free of non-iodophor antiseptic and non-iodophor therapeutic agent.

In an embodiment, the at least one non-iodophor, non-iodine therapeutic agent is an antifungal agent. Suitable antifungal agents include, for example, allylamines and azoles. In an embodiment, an antifungal agent includes, but is not limited to, tolnaftate, terbinafine, undecylenic acid, clioquinol, miconazole, miconazole nitrate, clorrinazole, tioconazole, nystatin, terconazoic, butoconazole nitrate, ciclopirox olamine, econazole nitrate, triacetin, flucytosine, haloprogin, and ketoconazole. In an embodiment, the composition of the invention can comprise PVP-I and DMSO and be free of non-iodophor antifungal agent.

In an embodiment, a composition comprises one or more naturopathic substances. Naturopathic substances include, but are not limited to, Punica Granatum (Pomegranate) Extract, Camellia Sinensis Leaf (Green Tea) Extract, Ascorbic Acid (Vitamin-C), Calendula Officinalis Extract, Glycrrhiza (Habra (Licorice) Extract, Allantoin, and Cucumis Sativus (Cucumber) Fruit Extract.

In an embodiment, a composition comprises DMSO, PVP-I, Punica Granatum (Pomegranate) Extract, Camellia Sinensis Leaf (Green Tea) Extract, Ascorbic Acid (Vitamin-C), Calendula Officinalis Extract, Glycrrhiza Glabra (Licorice) Extract, Allantoin, and Cucumis Sativus (Cucumber) Fruit Extract. In an embodiment, the composition of the invention comprises PVP-I and DMSO and is free of naturopathic agent.

In an embodiment, a composition comprises at least one solvent and/or penetrant. In an embodiment, a single component may function as both a solvent and a penetrant in the composition. In an embodiment, a composition comprises DMSO. In an aspect, DMSO functions as a penetrant for the active component. In an aspect, DMSO functions as a solvent. In yet another aspect, DMSO functions as both a solvent and a penetrant. In an embodiment, DMSO is the sole penetrant in a composition. In an embodiment, DMSO is the sole solvent in a composition. In an embodiment, DMSO is the sole penetrant and solvent in a composition.

In an embodiment, a composition comprises PVP-I and DMSO. In another embodiment, a composition consists of PVP-I and DMSO. In yet another embodiment, a composition consists essentially of PVP-I and DMSO. In an embodiment, a composition comprises PVP-I and DMSO and a co-solvent. In an embodiment, a composition comprises PVP-I, DMSO, a co-solvent and a gelling agent. In an embodiment, a composition consists of PVP-I and DMSO and a co-solvent. In an embodiment, a composition consists of PVP-I, DMSO, a co-solvent and a gelling agent. In any of the above-described compositions, the composition can be free of a second therapeutic agent or a second active ingredient. In any one of the above-described compositions, the composition can be free of solid or undissolved ingredient as particulate or in suspension in the composition.

One of skill in the art will understand, based on the disclosure herein, how to identify a penetrant useful for the compositions and methods encompassed herein. In an embodiment, a penetrant is one which is useful for enabling the composition to penetrate the thickened layer of mucus or skin, whether part of the natural anatomy or caused by the infection being treated. By way of a non-limiting example, methylsulfonylmethane may be used as a penetrant in a composition as encompassed herein.

In an embodiment, a composition comprises at least one co-solvent. In an embodiment, a composition comprises DMSO as a primary solvent, and further comprises at least one co-solvent. In an embodiment, water is a co-solvent. In an embodiment, a composition comprises DMSO as the primary solvent and water as a co-solvent. In an embodiment, a composition consists of DMSO as the primary solvent and water as the co-solvent. In another embodiment, a composition consists essentially of DMSO as the primary solvent and water as the co-solvent. In an embodiment, a composition comprises at least one co-solvent such as, but not limited to, water, or ethanol. In an embodiment, a co-solvent is one or more polar aprotic solvent. In an embodiment, a co-solvent is ethyl acetate. In an embodiment, a co-solvent is at least one of ethyl acetate, acetone, acetonitrile, tetrahydrofuran, methylene chloride, and dimethyl formamide. Examples of co-solvents include, but are not limited to, alcohols, silicones, polyglycols, glycols, and combinations thereof In an embodiment, a co-solvent is diethyleneglycol monoethyl ether (DGMEE). In an embodiment, a co-solvent is propylene glycol.

One of skill the art will understand the advantages and limitations of the use of co-solvents, based on the properties and physical effects of such potential co-solvents, in of the disclosure set forth herein.

In an embodiment, a composition comprises an iodophor and a penetrant, and further comprises water, wherein the water content is about at least 0.01%, about at least 0.02%, about at least 0.03%, about at least 0.04%, about at least 0.05%, about at least 0.06%, about at least 0.07%, about at least 0.08%, about at least 0.09%, about at least 0.1%, about at least 0.2%, about at least 0.3%, about at least 0.4%, about at least 0.5%, about at least 0.6%, about at least 0.7%, about at least 0.8%, about at least 0.9%, about at least 1.0%, about at least 1.5% about at least 2.0%, about at least 2.5%, about at least 5%, about at least 7.5%, about at least 10%, about at least 12.5%, or greater. In an embodiment, a composition comprises an iodophor and a penetrant, and further comprises water, wherein the water content is about less than 0.01%, about less than 0.02%, about less than 0.03%, about less than 0.04%, about less than 0.05%, about less than 0.06%, about less than 0,07%, about less than 0.08%, about less than 0.09%, about less than 0.1?4 about less than 0.2%, about less than 0.3%, about less than 0.4%, about less than 0.5%, about less than 0.6%, about less than 0.7%, about less than 0.8%, about less than 0.9%, about less than 1.0%, about less than 1.5%, about less than 2.0%, about less than 2.5%, about less than 5%, about less than 7.5%, about less than 10%, about less than 12.5%, or greater. In an embodiment, a composition comprises an iodophor and a penetrant, and further comprises water, wherein the water content is about 0.01% to about 12.5%, about 0.02% to about 10.0%, about 0.03% to about 7.5%, about 0.04% to about 5%, about 0.05% to about 2.5%, about 0.06% to about 2%, about 0.07% to about 1.5%, about 0.08% to about 1%, about 0.09% to about 0.9%, about 0.1% to about 0.8%, or about 0.2% to about 0.7%. In an aspect, the water may be derived from a component of the composition. In another aspect, the water may be specifically added to the composition.

In an embodiment, a composition comprises at least one excipient such as, but not limited to, sodium chloride, sodium dihydrogen phosphate monohydrate, disodium hydrogen phosphate anhydrous and water. The compositions encompassed herein will be understood to optionally include one or more other excipients as known to those skilled in the art. One of skill in the art will know how to identify such an excipient as useful in the present compositions and methods, for example, when such an excipient enhances the therapeutic effectiveness, stability, or potency of a composition or method.

### Dosages, Forms and Formulations

In an embodiment, a composition comprises a therapeutically effective amount of at least one therapeutic agent. The term “therapeutically effective amount” is used herein, unless otherwise indicated, to describe an amount of a compound which, in context, is used to produce or effect an intended therapeutic result. In an embodiment, the intended therapeutic result relates to the treatment of onychomycosis. In an embodiment, a therapeutically effective amount is that amount which is sufficient to treat an FHV infection, and the treatment of the FHV infection includes at least one of preventing or slowing the progression of the infection, preventing the spread of the infection, eradicating at least some of the infection, and eradicating the entire infection.

In an aspect, a therapeutically effective amount may be determined based on a single dosage or it may be determined based on multiple dosages of the composition. It will be understood that determination of the therapeutically effective amount may require trial and error, and may require adjustment of the dosage and or dosing regimen. Such therapeutic optimization and adjustment is encompassed by the methods encompassed herein.

The term “pharmaceutically acceptable”, as used herein with respect to a compound or composition, refers to a form of the compound or composition that can increase or enhance the solubility or availability of the compound in a subject, in order to promote or enhance the bioavailability of the compound or composition. In an aspect, the disclosure herein also encompasses pharmaceutically acceptable, hydrates, solvates, stereoisomers, or amorphous solids of the compounds and compositions embodied herein. A pharmaceutically acceptable composition further refers to a composition that is approvable by a competent governmental agency charged with granting approval of drugs or therapeutic agents for marketing within its jurisdiction. For example, a pharmaceutically acceptable composition marketed in the United States would be approvable by the U.S. Food and Drug Administration (FDA). For compositions used in veterinary application or treatment of animal disease or condition, a pharmaceutically acceptable composition or formulation refers to a composition or formulation that is approvable by a competent regulatory authority approving veterinary or animal drugs for marketing.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 1990, incorporated herein by reference). Except insofar as any conventional carrier is incompatible with the active ingredient or method of use, its use in the pharmaceutical compositions is contemplated.

Percentages set forth herein are (w/w), with respect to the specified component in the overall composition, unless otherwise indicated. For example, a composition comprising 0.25% PVP-I and 44% DMSO has 0.25% PVP-I by weight, and 44% by weight DMSO with respect to the total composition.

In an embodiment, a composition comprises an iodophor, PVP-I, in the range of about 0.01% to about 0.5%. In another embodiment, a composition comprises an iodophor in the range between 0.05% and 0.45%. In another embodiment, a composition comprises an iodophor in the range between 0.1% and 0.3%. In another embodiment, a composition comprises an iodophor in the range between 0.15% and 0.25%. In another embodiment, a composition comprises an iodophor in the range between 0.2% and 0.25%. In another embodiment, a composition comprises an iodophor in 0.25%.

In an embodiment, a composition comprises PVP-I in the range of about 0.01% to about 0.5%. In another embodiment, a composition comprises PVP-I in the range between 0.05% and 0.45%. In another embodiment, a composition comprises PVP-I in the range between 0.1% and 0.4%. In another embodiment, a composition comprises PVP-I in the range between 0.15% and 0.3%. In another embodiment, a composition comprises PVP-I in the range between 0.2% and 0.3%. in another embodiment, a composition comprises PVP-I in the range between 0.20% and 0.25%. In another embodiment, a composition comprises 0.25% PVP-I.

In an embodiment, anhydrous DMSO is used in a composition. In an embodiment, substantially anhydrous DMSO is used in a composition. It will be understood by one of skill in the art that DMSO can be produced and/or obtained in differing grades, and that one of the variables among DMSO preparations of different grades is the water content. By way of example, DMSO may be completely anhydrous (also referred to herein simply as “anhydrous”), substantially anhydrous, or may contain water to a measurable degree. It will be understood that the amount of measurable water in a DMSO preparation may vary based on limitations of the instrumentation and techniques used to make such measurements. In an embodiment, DMSO that is not completely anhydrous may be substantially anhydrous and contain water at a level below levels of detectability. In an embodiment, DMSO that is not completely anhydrous may contain water, wherein the water content is about at least 0.01%, about at least 0.02%, about at least 0.03%, about at least 0.04%, about at least 0.05%, about at least 0.06%, about at least 0.07%, about at least 0.08%, about at least 0.09%, about at least 0.1%, about at least 0.2%, about at least 0.3%, about at least 0.4%, about at least 0.5%, about at least 0.6%, about at least 0.7%, about at least 0.8%, about at least 0.9%, about at least 1.0%, about at least 1.5%, about at least 2.0%, about at least 2.5%, about at least 5%, about at least 7.5%, about at least 10%, about at least 12.5%, or greater. In an embodiment, DMSO that is not completely anhydrous may contain water, wherein the water content is about less than 0.01%, about less than 0.02%, about less than 0.03%, about less than 0.04%, about less than 0.05%, about less than 0.06%, about less than 0.07%, about less than 0.08%, about less than 0.09%, about less than 0.1%, about less than 0.2%, about less than 0.3%, about less than 0.4%, about less than 0.5%, about less than 0.6%, about less than 0.7%, about less than 0.8% about less than 0.9%, about less than 1.0%, about less than 1.5%, about less than 2.0%, about less than 2.5%, about less than 5%, about less than 7.5%, about less than 10%, about less than 12.5%, or greater. It will be understood that DMSO may contain one or more other impurities in addition to water.

In an embodiment, a composition comprises at least one of United States Pharmacopeial Convention (USP) grade DMSO, Active Pharmaceutical Ingredient (API) grade DMSO, analytical grade DMSO, and American Chemical Society (ACS) Spectrophotometric grade DMSO. In an embodiment, a composition comprises DMSO having <0.1% water by KF titration and >99.9% determined on an anhydrous basis.

In an embodiment, a composition comprises DMSO in the range of 25% to 99.99%. In an embodiment, a composition comprises DMSO in the range of greater than 25% to 99.99%. In another embodiment, a composition comprises DMSO in the range of 30% and 99.9%. In another embodiment, a composition comprises DMSO in the range of greater than 30% and 99.9%. In another embodiment, a composition comprises DMSO in the range of 33% and 99.9%. in another embodiment, a composition comprises DMSO in the range of 35% and 99.9%. In another embodiment, a composition comprises DMSO in the range of 40% and 99.9%. In another embodiment, a composition comprises DMSO in the range of 50% and 99.9%. In another embodiment, a composition comprises DMSO in the range of 30% and 90%. In another embodiment, a composition comprises DMSO in the range of 35% and 75%. In another embodiment, a composition comprises DMSO in the range of 40% and 60%, and in yet another embodiment, between 40% and 50%. In an embodiment, a composition comprises DMSO in weight percent of about 44%, In an embodiment, a composition comprises DMSO in weight percent of 44%.

In an embodiment, a composition comprises DMSO but does not comprise any additional solvent (e.g., co-solvent) or penetrant. In another embodiment, a composition comprises DMSO in the range of about 30% to 99.99% and further comprises at least one co-solvent in the range of 0.01% to about 70%. In an embodiment, a composition comprises DMSO and further comprises at least one co-solvent in the range of about 0.1% to about 50%. In an aspect, water is a co-solvent. In an embodiment, a composition comprises DMSO, water, and at least one additional co-solvent.

### Stability—Measured as Percent Iodine Remaining

In one embodiment, the formulations are stable at room temperature 25° C. for at least 6 months, 12 months, 18 months and 24 months. Stability is defined as where the final PVP-I concentration is at least 85% of the labeled concentration (e.g. if the label is 2% PVP-I providing for 0.2% iodine, therefore 90% would be 0.18 elemental iodine).

In one embodiment, the formulations are stable at room temperature 2-8° C. for at least 6 months, 12 months, 18 months and 24 months. Stability is defined as where the final PVP-I concentration is at least 90% of the labeled concentration (e.g. if the label is PVP-I providing for 0.2% iodine, therefore 90% would be 0.18 elemental iodine).

In one embodiment, the formulations are stable at room temperature −10 to −25° C. for at least 6 months, 12 months, 18 months and 24 months. Stability is defined as where the final PVP-I concentration is at least 90% of the labeled concentration (e.g. if the label is 2% PVP-I providing for 0.2% iodine, therefore 90% would be 0.18 elemental iodine).

In one embodiment, the formulations are stable at room temperature 15-30° C. for at least 6 months, 12 months, 18 months and 24 months. Stability is defined as where the final PVP-I concentration is at least 90% of the labeled concentration (e.g. if the label is 2% PVP-I providing for 0.2% iodine, therefore 90% would be 0.18 elemental iodine).

In one embodiment, the formulations are stable at room temperature 40° C. for at least 1 months, 3 months, 6 months, 12 months, 18 months and 24 months. Stability is defined as where the final PVP-I concentration is at least 90% of the labeled concentration (e.g. if the label is 2% PVP-I providing for 0.2% iodine, therefore 90% would be 0.18 elemental iodine.

### Methods of Preparation and Use

It is known to one of skill in the art that PVP-I aqueous solutions are difficult to stabilize at low PVP-I concentrations over a long period of time. By way of a non-limiting example, at concentrations of PVP-I less than about 0.7% (w/w, aqueous), PVP-I aqueous solutions rapidly decay to yield complex mixtures of iodinated and iodine-free constituents. As described herein, it was surprisingly found that in the aprotic DMSO solvent system encompassed by the disclosure set forth herein, PVP-I solutions as low as 0.1% can be easily prepared and maintained as stable compositions for long periods of time. Also as described herein, hydrated DMSO solutions prepared from aqueous PVP-I demonstrate increased stability was noted for the PVP-I component.

In an embodiment, a composition comprises dry, solid or powdered PVP-I dissolved or suspended in a composition comprising or consisting of DMSO. In another embodiment, DMSO is added to an aqueous preparation comprising or consisting of PVP-I. Based on the disclosure herein, one of skill in the art will understand how to prepare a composition to arrive at the desired amounts of iodine, iodophor, and DMSO, among other possible components of the compositions encompassed herein.

By way of a non-limiting example, a therapeutically-effective pharmaceutical composition is prepared using solid PVP-I, which is dissolved or suspended in DMSO. In an aspect, the composition is anhydrous. In an aspect, the composition is substantially anhydrous. In another embodiment, DMSO can be added to aqueous solutions of PVP-I to prepare a therapeutically-effective pharmaceutical composition. In an embodiment, DMSO is used in the range of 50%-99% as a co-solvent with water. In an embodiment, a formulation includes one or more excipients. By way of a non-limiting example, excipients include, but are not limited to, sodium chloride, sodium dihydrogen phosphate monohydrate, disodium hydrogen phosphate anhydrous and water, as well as others known to those skilled in the art.

In an embodiment, a composition is prepared by adding 0.25% PVP-I (w/v, aqueous) to pure DMSO q.s. to yield a resulting solution of 0.25% PVP-I (w/w) with DMSO. In another embodiment, compositions are prepared by dissolving solid PVP-I in pure DMSO q.s to obtain any of 0.1%, 0.15%, 0.2%, 0.25%. 0.3%, 0.35%. 0.4%, 0.45%. or 0.5% PVP-I (w/w), with DMSO as the solvent. In yet another embodiment, compositions are prepared by dissolving solid PVP-I in pure DMSO q.s. to obtain any composition set forth, described, and/or encompassed herein. Similar compositions comprising aqueous PVP-I (with and without excipients commonly used and/or known in the art) and DMSO can be prepared from a stock 10% PVP-I aqueous solution and pure DMSO. It will be understood by the skilled artisan, however, that any starting composition of PVP-I, solid or liquid, may be used when the appropriate dilutions and adjustments are made to result in the desired final PVP-I concentration.

In an embodiment, it is particularly useful for the case of FHV infections that stable, anhydrous compositions that contain between 0.01%-0.25% PVP-I can be prepared in pure USP grade DMSO solvents.

It will be understood, based on the disclosure set forth herein, in view of the skill in the art, that specific dosage for compounds and compositions encompassed herein may be determined empirically through clinical and/or pharmacokinetic experimentation, and that such dosages may be adjusted according to pre-specified effectiveness and/or toxicity criteria. It will also be understood that a specific dosage and treatment regimen for any particular patient will depend upon a variety of factors, including the activity of the specific compounds employed, the characteristics of the patient, drug combination, the judgment of the treating physician and the nature and severity of the particular disease or condition being treated. Dosing of a gel formulation, for example, comprises administration of an approximately ½ inch ribbon directly to the surface of the eye, preferably under the eyelid, one or two times per day until the infection is clinically subsided. Dosing of the liquid formulation comprises administration of 1-5 drops, typically 2-3 drops, of the composition directly to the eye.

In an embodiment, a method of treating a subject having an FHV infection includes administration of a composition set forth, described, and/or encompassed herein to treat the FHV infection, and the treatment of the FHV infection includes at least one of preventing or slowing the progression of the infection, preventing the spread of the infection, eradicating at least some of the infection, and eradicating the entire infection.

In an embodiment, a therapeutic composition is administered on a schedule once a day. In an embodiment, a therapeutic composition is administered twice a day. In an embodiment, a therapeutic composition is administered three times a day, four times a day, five times a day, or more. In an embodiment, a therapeutic composition is administered less frequently than once a day. In an embodiment, a therapeutic composition is administered once every two days, once every three days, once every four days, once every five days, once every six days, or once every seven days. In an embodiment, a therapeutic composition is administered less frequently than once a week. In an embodiment, a therapeutic composition is administered once a month. In an embodiment, a therapeutic composition is administered twice a month.

In an embodiment, a therapeutic dosing regimen is continued for at least one day, at least two days, at least three days, at least four days, at least five days, at least six days, or at least seven days. In an embodiment, a therapeutic dosing regimen is continued for at least one week, at least two weeks, at least three weeks, at least four weeks, at least six weeks, at least eight weeks, at least ten weeks, at least twelve weeks, at least fourteen weeks, or at least sixteen weeks. In an embodiment, a therapeutic dosing regimen is continued for at least one month, at least two months, at least three months, at least four months, at least five months, at least six months, at least nine months, or at least twelve months.

The invention is further described by the following examples. In an aspect, the following examples demonstrate effective and/or successful treatment of the identified conditions using compositions and methods encompassed by the present disclosure. It should be recognized that variations based on the inventive features are within the skill of the ordinary artisan, and that the scope of the invention should not be limited by the examples. To properly determine the scope of the invention, an interested party should consider the claims herein, and any equivalent thereof In addition, all citations herein are incorporated by reference, and unless otherwise expressly stated, all percentages are by weight.

### Examples of Felines, Canines, and Equines

The following prophetic examples illustrate the use of a composition, as described herein, in accordance with a method of the subject invention. The results of the described treatments are within the scope of the subject invention as contemplated and conceived, and thus can be fully realized by performing the method steps described, yet are unexpected in view of the prior art.

It will be appreciated by those skilled in the art that changes could be made to the exemplary embodiments shown and described above without departing from the broad inventive concept thereof. It is understood, therefore, that the disclosure herein is not limited to the exemplary embodiments shown and described, but it is intended to cover modifications within the spirit and scope of the present invention as defined by the claims. For example, specific features of the exemplary embodiments may or may not he part of the claimed invention and features of the disclosed embodiments may be combined. Unless specifically set forth herein, the terms “a”, “an” and “the” are not limited to one element and should be read as meaning “at least one”.

It is to be understood that at least sonic of the descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent the method does not rely on the particular order of steps, the particular order of the steps should not be construed as limitation on the claims.

The claims directed to the method of the present invention should not be limited to the performance of their steps in the order written, and one skilled in the art can readily appreciate that the steps may be varied and still remain within the spirit and scope of the present invention.

A domestic cat presents with visible, unilateral or bilateral infection of the eye. Following a diagnosis that the infection results from Feline Herpesvirus (FHV), a dosing regimen can be employed whereby an approximately ½-inch ribbon of a composition comprising 0.25% PVP-I and 44% DMS) can be administered directly to the surface of the infected eye or eyes, preferably between corneal surface and the lower eyelid. The composition can be administered one time per day or two times per day for a period of about three days, up to a period of about one week. The infection will visibly subside within two days to about one week. The described use of the composition can provide better efficacy and faster treatment results for (subsiding of) the infection than without use of the composition.

Advantageously, the composition is efficacious without harmful side effects and without irritation or burning sensation to the eye of the animal.

A domestic cat presents with visible, unilateral infection of the cornea. Following a diagnosis that the infection results from Feline Herpesvirus (FHV) related corneal ulceration, a dosing regimen can be employed whereby an approximately ½-inch ribbon of a composition comprising 0.25% PCP-I and 44% DMSO can be administered directly to the surface of the infected eye or eyes, preferably between corneal surface and the lower eyelid, The composition can be administered six times per day for a period up to two weeks. The infection will visibly subside within about one week. The described use of the composition can provide better efficacy and faster treatment results for (subsiding of) the infection than without use of the composition.

A domestic cat presents with visible, unilateral or bilateral keratoconjunctivitis sicca. Following a diagnosis that the infection results from Feline Herpesvirus (FHV), a dosing regimen can be employed whereby an approximately ½-inch ribbon of a composition comprising 0.25% PCP-I and 44% DMSO can be administered directly to the surface of the infected eye or eyes, preferably between corneal surface and the lower eyelid. The composition can be administered one time per day or two times per day for a period of about one week, up to a period of one month. The inflammation will visibly subside within one week to about one month. The described use of the composition can provide better efficacy and faster treatment results for (subsiding of) the inflammation than without use of the composition.

A domestic cat presents with visible, unilateral non-mobile black lesion on the cornea. Following a diagnosis that the mass results from Feline Herpesvirus (FHV) related corneal sequestration, a dosing regimen can be employed whereby an approximately ½-inch ribbon of a composition comprising 0.25% PCP-I and 44% DMSO can be administered directly to the surface of the infected eye or eyes, preferably between conical surface and the lower eyelid. The composition can be administered four times per day for a period up to two weeks. The mass will visibly subside within about one week. The described use of the composition can provide better efficacy and faster treatment results for (subsiding of) the mass or infection than without use of the composition,

A domestic cat presents with visible, unilateral eye discomfort, pain and discharge. Ophthalmoscopy reveals a corneal dendritic ulceration that stains with fluorescein. The patient receives a diagnosis of Feline Herpesvirus (FHV) epithelial keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period often days. After three days of treatment, the dendritic ulceration begins to re-epithelialize and any surrounding corneal inflammation abates. The infection is completely healed within 5 days as evidenced by a normal ocular inspection and the patient returns to normal behavior and activity.

A domestic cat presents with visible, bilateral eye discomfort, pain, redness and discharge. Ophthalmoscopy reveals bilateral erythematous conjunctiva with mild chemosis and discharge. The patient receives a diagnosis of Feline Herpesvirus (FHV) related conjunctivitis and a dosing regimen is employed whereby an approximately ½inch ribbon of a composition comprising 0.25% PCP-I and 44% DMSO is administered directly to the surface of the infected eye preferably between corneal surface and the lower eyelid. The composition is administered four times per day per for a period of ten days. After three days of treatment, both the chemosis and erythema begin to improve. By the fifth day, the residual inflammation abates and the eye ocular examination is unremarkable. The patient continues the prescribed medicine for the full course of ten days in order to prevent spread to others in the litter.

A domestic cat presents with visible, bilateral eye discomfort, pain, redness and discharge after being treated for Feline Herpesvirus (FHV) related conjunctivitis with a topical antiviral medicine. Ophthalmoscopy reveals bilateral erythematous conjunctiva with moderate chemosis and green purulent discharge. The patient receives a diagnosis of bacterial conjunctivitis related to bacterial overgrowth after treatment for Feline Herpesvirus (FHV) related conjunctivitis. For treatment, a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day for a period of seven days. After three days of treatment, the purulent discharge has resolved, however, conjunctival inflammation persists. By the Fifth day, the conjunctiva is clear, without chemosis or erythema and the remaining ocular examination is unremarkable. The patient continues the prescribed medicine for the full course of ten days in order to prevent spread to others in the litter.

A domestic cat presents with visible, unilateral eye discomfort, pain and erythema. Ophthalmoscopy reveals a mild conjunctivitis and an intact corneal epithelium. In the corneal stroma a disciform infiltrate is present along with endothelial precipitates. The patient receives a diagnosis of Feline Herpesvirus (FHV) related stromal keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of two weeks. After five days of treatment, the stromal infiltrate begins to lessen in density. There are no signs of breakthrough epithelial keratitis. After ten clays, the infiltrate has continued to decrease and the endothelial changes and conjunctivitis have resolved. The infection is completely healed within two weeks as evidenced by normal ocular inspection and the patient returns to normal behavior and activity.

A domestic cat presents with visible, unilateral eye discomfort, pain and photophobia. Ophthalmoscopy reveals an intact cornea and mild perilimbal flush. Inspection of the anterior chamber reveals 2+ cells and flare without hypopyon. The patient receives a diagnosis of Feline Herpesvirus (FHV) related uveitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered six times per day per for a period of ten days. After two days of treatment, the anterior chamber cell and flare reaction has decreased to 1+ and the patient's photophobia has abated. Accordingly, the composition is now administered in a tapered fashion until it is utilized only once daily by the tenth day. Examination on the last day of treatment reveals a clear cornea, white conjunctiva and an anterior chamber that is both deep and quiet. The patient returns to normal behavior and activity.

A domestic cat presents with visible, unilateral eye discomfort, pain and discharge after being struck in the eye by a tree branch. Ophthalmoscopy reveals a corneal ulceration with crenate, feathered borders measuring 2×2 mm. A satellite corneal infiltrate is also present. Anterior chamber reveals 1+ cell and flare. The patient receives a diagnosis of fungal corneal ulceration and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of one month. After one week of treatment, the ulceration begins to re-epithelialize, diminish in size and any surrounding corneal inflammation abates. The infection is completely healed within three weeks as evidenced by a normal ocular inspection and the patient returns to normal behavior and activity.

A domestic kitten presents with visible, unilateral eye discomfort, inability to open the eye, pain, redness and discharge at birth. Ophthalmoscopy reveals conjunctival inflammation with moderate chemosis and punctate epithelial erosions of the corneal surface. There is also a mucopurulent discharge present. The patient receives a diagnosis of Feline Herpesvirus (FHV) related ophthalmia neonatorium. Accordingly, a dosing regimen is employed whereby an approximately ½-inch ribbon of a composition comprising 0.25% PCP-I and 44% DMSO is administered directly to the surface of the infected eye preferably between conical surface and the lower eyelid. The composition is administered four times per day per for a period of one week. After two days of treatment, the kitten is able to open the eye of her own volition. After five days of treatment, the discharge has ceased and the conjunctival inflammation has improved significantly. The infection is completely healed within seven days as evidenced by a normal ocular inspection and the patient returns to the litter, exhibiting normal behavior and activity.

A domestic cat presents with visible, unilateral eye discomfort, pain and erythema with discharge. On visual inspection the affected eye appears pink/grey in color. Ophthalmoscopy reveals a mild conjunctivitis and an intact corneal epithelium, however, the peripheral limbus reveals elevated nodules with trace perilimbal infiltrate. The patient receives a diagnosis of Feline Herpesvirus (FHV) related eosinophilic keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of two weeks. After five days of treatment, the peripheral limbal infiltrate begins to lessen in density. There are no signs of breakthrough epithelial keratitis. After ten days, the infiltrate has continued to decrease and the conjunctivitis has resolved. The infection is completely healed within two weeks as evidenced by normal ocular inspection and coloration. The patient returns to normal behavior and activity.

A domestic cat presents with visible, unilateral eye discomfort, pain and discharge after a trauma received while playing. Ophthalmoscopy reveals a suppurative corneal ulceration with 30% stromal loss measuring 3×3 mm. Anterior chamber reveals 1+ cell and flare, but is negative for hypopyon. The patient receives a diagnosis of trauma related bacterial corneal ulceration and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of one month. After one week of treatment, the ulceration begins to re-epithelialize, diminish in size and any surrounding corneal inflammation abates. The infection is completely healed within three weeks as evidenced by a normal ocular inspection and the patient returns to normal behavior and activity.

A domestic cat presents with visible, bilateral eye discomfort, pain, redness and discharge after being in contact with other infected cats. Ophthalmoscopy reveals bilateral erythematous conjunctiva with moderate chemosis and discharge. The patient receives a diagnosis of chlamydia or mycoplasma related conjunctivitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered two times per day per for a period of one week. After three days of treatment, both the chemosis and erythema begin to improve. By the fifth day, the residual inflammation abates and the eye ocular examination is unremarkable. The patient continues the prescribed medicine for the full course of seven days in order to prevent spread to others in the cattery.

A domestic dog presents with visible, unilateral eye discomfort, pain and discharge. Ophthalmoscopy reveals a corneal dendritic ulceration that stains with fluorescein. The patient receives a diagnosis of Canine Herpesvirus (CHV) epithelial keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of ten days. After three days of treatment, the dendritic ulceration begins to re-epithelialize and any surrounding corneal inflammation abates. The infection is completely healed within 5 days as evidenced by a normal ocular inspection and the patient returns to normal behavior and activity.

A domestic dog presents with visible, unilateral eye discomfort, pain and discharge after being struck in the eye by vegetative matter. Ophthalmoscopy reveals a corneal ulceration with crenate, feathered borders measuring 2×2 mm. A satellite corneal infiltrate is also present. Anterior chamber reveals 1+ cell and flare. The patient receives a diagnosis of fungal corneal ulceration and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of one month. After one week of treatment, the ulceration begins to re-epithelialize, diminish in size and any surrounding corneal inflammation abates. The infection is completely healed within three weeks as evidenced by a normal ocular inspection and the patient returns to normal behavior and activity.

A domestic dog presents with visible, bilateral eye discomfort, pain, redness and discharge after being treated for Canine Herpesvirus (CHV) related conjunctivitis with a topical antiviral medicine. Ophthalmoscopy reveals bilateral erythematous conjunctiva with moderate chemosis and green purulent discharge. The patient receives a diagnosis of bacterial conjunctivitis related to bacterial overgrowth after treatment for Canine Herpesvirus (CHV) related conjunctivitis. For treatment, a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day for a period of seven days. After three days of treatment, the purulent discharge has resolved, however, conjunctival inflammation persists. By the fifth day, the conjunctiva is clear, without chemosis or erythema and the remaining ocular examination is unremarkable. The patient continues the prescribed medicine for the full course of ten days in order to prevent spread to others in the kennel.

A domestic dog presents with visible, unilateral eye discomfort and excoriations of the eyelid. Ophthalmoscopy reveals a healthy conjunctival examination and a cornea without ulceration. The eyelid inspection reveals small ulcers at the eyelid margin in conjunction with lid margin edema, erythema, and eyelash matting and dandruff. The patient receives a diagnosis of Canine Herpesvirus (CHV) related blepharitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of two weeks. After three days of treatment, the eyelid ulceration begins to re-epithelialize and surrounding eyelid inflammation abates. The infection is completely healed within two weeks as evidenced by a normal ocular inspection and resolution of the eyelid and eyelash inflammation. The patient returns to normal behavior and activity.

A domestic dog presents with visible, bilateral eye discomfort, pain, redness and discharge. Ophthalmoscopy reveals bilateral erythematous conjunctiva with mild chemosis and discharge. The patient receives a diagnosis of Canine Herpesvirus (CHV) related conjunctivitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of ten days. After three days of treatment, both the chemosis and erythema begin to improve. By the fifth day, the residual inflammation abates and the eye ocular examination is unremarkable. The patient continues the prescribed medicine for the full course of ten days in order to prevent spread to others in the litter.

A domestic dog presents with visible, bilateral eye discomfort, pain, redness and discharge after being in contact with other infected dogs. Ophthalmoscopy reveals bilateral erythematous conjunctiva with moderate chemosis and discharge. The patient receives a diagnosis of bacterial conjunctivitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered two times per day per for a period of one week. After three days of treatment, both the chemosis and erythema begin to improve. By the fifth day, the residual inflammation abates and the eye ocular examination is unremarkable. The patient continues the prescribed medicine for the full course of seven days in order to prevent spread to others in the kennel.

A domestic dog presents with visible, bilateral ocular erythema and mild mucous discharge. Ophthalmoscopy reveals punctate corneal erosions, mild conjunctivitis, and a decreased tear break up time. The patient receives a diagnosis of Canine Herpesvirus (CHV) related keratoconjunctivitis sicca and a dosing regimen is employed whereby an approximately ½-inch ribbon of a composition comprising 0.25% PCP-I and 44% DMSO can be administered directly to the surface of the infected eye or eyes, preferably between conical surface and the lower eyelid. The composition is administered two times per day for a period of about one week, up to a period of one month. After one week of treatment, the inflammation begins visibly subside as evidenced by an improved tear break up time. After 3 weeks, the conjunctival erythema is resolved and the remaining ocular examination is within normal limits. The patient returns to normal behavior and activity.

A domestic dog presents with visible, unilateral eye discomfort, pain and clear discharge. Ophthalmoscopy reveals mild conjunctival injection and diffuse, punctate epithelial erosions present over the corneal surface. The anterior chamber is deep and quiet. The patient receives a diagnosis of Canine Herpesvirus (CHV) non-ulcerative keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of ten days. After three days of treatment, the corneal erosions begin to heal and any surrounding corneal inflammation abates. The infection is completely healed within 5 days as evidenced by a normal ocular inspection and the patient returns to normal behavior and activity.

A domestic dog presents with visible, unilateral eye discomfort, pain and discharge after a trauma received while playing. Ophthalmoscopy reveals a suppurative corneal ulceration with 30% stromal loss measuring 3×3 mm. Anterior chamber reveals 1+ cell and flare, but is negative for hypopyon. The patient receives a diagnosis of trauma related bacterial corneal ulceration and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of one month. After one week of treatment, the ulceration begins to re-epithelialize, diminish in size and any surrounding corneal inflammation abates. The infection is completely healed within three weeks as evidenced by a normal ocular inspection and the patient returns to normal behavior and activity.

A domestic dog presents with visible, unilateral eye discomfort, pain and erythema with discharge. On visual inspection, the affected eye appears pink/grey in color. Ophthalmoscopy reveals a mild conjunctivitis and an intact corneal epithelium, however, the peripheral limbus reveals elevated pink or gray nodules with trace perilimbal white infiltrates. The patient receives a diagnosis of Canine Herpesvirus (CHV) related eosinophilic keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of two weeks. After five days of treatment, the peripheral limbal infiltrate begins to lessen in density. There are no signs of breakthrough epithelial keratitis. After ten days, the infiltrate has continued to decrease and the conjunctivitis has resolved. The infection is completely healed within two weeks as evidenced by normal ocular inspection and coloration. The patient returns to normal behavior and activity.

A thoroughbred racehorse presents with visible, bilateral eye discomfort, pain, redness and discharge after being in contact with other infected horses. Ophthalmoscopy reveals bilateral erythematous conjunctiva with moderate chemosis and clear discharge. There are a few staining punctate conical epithelial erosions. The patient receives a diagnosis of Equine Herpesvirus (EHV) related epidemic keratoconjunctivitis and a dosing regimen is employed whereby an approximately ½-inch ribbon of a composition comprising 0.25% PCP-I and 44% DMSO is administered directly to the surface of the infected eye preferably between conical surface and the lower eyelid. The composition is administered two times per day per for a period of two weeks. After three days of treatment, both the chemosis and erythema begin to improve. By the fifth day, the residual inflammation abates and the eye ocular examination is unremarkable. The patient continues the prescribed medicine for the full course of seven days in order to prevent spread to others.

A domestic horse presents with visible, bilateral eye discomfort, pain, redness and discharge. Ophthalmoscopy reveals bilateral erythematous conjunctiva with moderate chemosis and clear discharge. There are a few staining punctate corneal epithelial erosions. The patient receives a diagnosis of Equine Herpesvirus (EHV) related keratoconjunctivitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered two times per day per for a period of two weeks. After three days of treatment, both the chemosis and erythema begin to improve. By the fifth day, the residual inflammation abates and the eye ocular examination is unremarkable. The patient continues the prescribed medicine for the full course of seven days in order to decrease viral shedding.

A thoroughbred racehorse presents with visible, unilateral eye discomfort, pain and erythema with discharge. On visual inspection, the affected eye appears pink/grey in color. Ophthalmoscopy reveals a mild conjunctivitis and an intact corneal epithelium, however, the peripheral limbus reveals elevated pink or gray nodules with trace perilimbal white infiltrates. The patient receives a diagnosis of Equine Herpesvirus (EHV) related eosinophilic keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of two weeks. After five days of treatment, the peripheral limbal infiltrate begins to lessen in density. There are no signs of breakthrough epithelial keratitis. After ten days, the infiltrate has continued to decrease and the conjunctivitis has resolved. The infection is completely healed within two weeks as evidenced by normal ocular inspection and coloration. The patient returns to normal behavior and activity.

A thoroughbred racehorse presents with poor appetite and decreased visual function of the left eye. On visual inspection, the eye and adenexa are normal in appearance. Ophthalmoscopy reveals a normal anterior segment; however, retinal examination reveals multifocal chorioretinal infiltrates. The patient receives a diagnosis of Equine Herpesvirus (EHV) related multifocal chorioditis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of two weeks. After one week, the multifocal lesions begin to resolve and the patient begins to eat and behave normally. The infection is completely healed within two weeks as evidenced by normal ocular inspection and retinal assessment. The patient continues normal behavior and activity.

A domestic horse presents with visible, unilateral eye discomfort, pain and discharge. Ophthalmoscopy reveals a corneal dendritic ulceration that stains with fluorescein. The patient receives a diagnosis of Equine Herpesvirus (FHV) epithelial keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of ten days. After three days of treatment, the dendritic ulceration begins to re-epithelialize and any surrounding corneal inflammation abates. The infection is completely healed within 5 days as evidenced by a normal ocular inspection and the patient returns to normal behavior and activity.

A thoroughbred racehorse presents with visible, unilateral eye discomfort, pain and discharge after being struck in the eye by a tree branch. Ophthalmoscopy reveals a corneal ulceration with crenate, feathered borders measuring 2×2 mm. A satellite corneal infiltrate is also present. Anterior chamber reveals 1+ cell and flare. The patient receives a diagnosis of fungal corneal ulceration and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of one month. After one week of treatment, the ulceration begins to re-epithelialize, diminish in size and any surrounding corneal inflammation abates. The infection is completely healed within three weeks as evidenced by a normal ocular inspection and the patient returns to normal behavior and activity.

A thoroughbred racehorse presents with visible, unilateral eye discomfort, pain and discharge after a trauma received while racing. Ophthalmoscopy reveals a suppurative conical ulceration with 30% stromal loss measuring 3×3 mm. Anterior chamber reveals 1+ cell and flare, but is negative for hypopyon. The patient receives a diagnosis of trauma related. bacterial corneal ulceration and a dosing regimen is employed whereby an approximately ½-inch ribbon of a composition comprising 0.25% PCP-I and 44% DMSO is administered directly to the surface of the infected eye preferably between conical surface and the lower eyelid. The composition is administered four times per day per for a period of one month. After one week of treatment, the ulceration begins to re-epithelialize, diminish in size and any surrounding corneal inflammation abates. The infection is completely healed within three weeks as evidenced by a normal ocular inspection and the patient returns to normal behavior and activity.

A domestic horse presents with visible, unilateral eye discomfort and excoriations of the eyelid. Ophthalmoscopy reveals a healthy conjunctival examination and a cornea without ulceration. The eyelid inspection reveals small ulcers at the eyelid margin in conjunction with lid margin edema, erythema, and eyelash matting and dandruff. The patient receives a diagnosis of Equine Herpesvirus (EHV) related blepharitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered two times per day per for a period of two weeks. After three days of treatment, the eyelid ulceration begins to re-epithelialize and surrounding eyelid inflammation abates. The infection is completely healed within two weeks as evidenced by a normal ocular inspection and resolution of the eyelid and eyelash inflammation. The patient returns to normal behavior and activity.

A domestic horse presents with visible, unilateral eye discomfort, pain and clear discharge. Ophthalmoscopy reveals mild conjunctival injection and diffuse, punctate epithelial erosions present over the corneal surface. The anterior chamber is deep and quiet. The patient receives a diagnosis of Equine Herpesvirus (EHV) non-ulcerative keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of ten days. After three days of treatment, the corneal erosions begin to heal and any surrounding corneal inflammation abates. The infection is completely healed within 5 days as evidenced by a normal ocular inspection and the patient returns to normal behavior and activity.

A thoroughbred racehorse presents with visible, bilateral ocular erythema and mild mucous discharge. Ophthalmoscopy reveals punctate corneal erosions, mild conjunctivitis, and a decreased tear break up time. The patient receives a diagnosis of Equine Herpesvirus (EHV) related keratoconjunctivitis sicca and a dosing regimen is employed whereby an approximately ½-inch ribbon of a composition comprising 0.25% PCP-I and 44% DMS) can be administered directly to the surface of the infected eye or eyes, preferably between corneal surface and the lower eyelid. The composition is administered two times per day for a period of about one week, up to a period of one month. After one week of treatment, the inflammation begins visibly subside as evidenced by an improved tear break up time. After 3 weeks, the conjunctival erythema is resolved and the remaining ocular examination is within normal limits. The patient returns to normal behavior and activity.

A thoroughbred racehorse presents with visible, bilateral eye discomfort, pain, redness and discharge after being treated for Equine Herpesvirus (EHV) related conjunctivitis with a topical antiviral medicine. Ophthalmoscopy reveals bilateral erythematous conjunctiva with moderate chemosis and green purulent discharge. The patient receives a diagnosis of bacterial conjunctivitis related to bacterial overgrowth after treatment for Equine Herpesvirus (EHV) related conjunctivitis. For treatment, a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered two times per day for a period of seven days. After three days of treatment, the purulent discharge has resolved, however, conjunctival inflammation persists. By the fifth day, the conjunctiva is clear, without chemosis or erythema and the remaining ocular examination is unremarkable. The patient continues the prescribed medicine for the full course of ten days in order to prevent spread to others in the kennel.

A thoroughbred racehorse presents with visible, unilateral eye discomfort, pain and erythema. Ophthalmoscopy reveals a mild conjunctivitis and an intact corneal epithelium. In the corneal stroma a disciform infiltrate is present along with endothelial precipitates. The patient receives a diagnosis of Equine Herpesvirus (EHV) related stromal keratitis and a dosing regimen is employed whereby an approximately ½-inch ribbon of a composition comprising 0.25% PCP-I and 44% DMSO is administered directly to the surface of the infected eye preferably between corneal surface and the lower eyelid. The composition is administered four times per day per for a period of two weeks. After five days of treatment, the stromal infiltrate begins to lessen in density. There are no signs of breakthrough epithelial keratitis. After ten days, the infiltrate has continued to decrease and the endothelial changes and conjunctivitis have resolved. The infection is completely healed within two weeks as evidenced by normal ocular inspection and the patient returns to normal behavior and activity.

1. A method for treating herpesvirus infection in an eye of an animal, said method comprising:
providing a pharmaceutically acceptable composition comprising 0.01% to 5.0% povidone-iodine (PVP-I) and greater than 30% DMSO; and
administering an effective amount of the composition to the eye of the animal.

2. The method of claim 1 wherein the composition comprises 0.25% PVP-I.

3. The method of claim 1 wherein the composition comprises 44% DMSO.

4. The method of claim 1 wherein the composition further comprises a co-solvent.

5. The method of claim 1 wherein the composition comprises a gelling agent in an amount effective for forming a stable gel composition.

6. The method of claim 5 wherein the gelling agent is selected from gum, agar, carrageenan, petrolatum, and a cellulosic polymer.

7. The method of claim 5 wherein the gelling agent is a cellulosic polymer.

8. The method of claim 7 wherein the cellulosic polymer is hydroxyethylcellulose.

9. The method of claim 1 wherein the animal is non-human.

10. The method of claim 9 wherein the animal is feline and the herpesvirus is feline herpesvirus (FHV).

11. The method of claim 9 wherein the animal is canine and the herpesvirus is canine herpesvirus (CHV).

12. The method of claim 9 wherein the animal quine and the herpesvirus is equine herpesvirus (EHV).

13. The method of claim 1 wherein the herpesvirus infection is herpes keratitis.

14. A pharmaceutically acceptable composition for use in treating herpesvirus infection in an of an animal, said composition comprising:
from 0.01% to 5.0% povidone-iodine (PVP-I) and greater than 30% DMSO;
wherein the composition is administered in an effective amount to the eye of the animal to ameliorate or eliminate the infection.

15. The composition of claim 14 wherein the composition comprises 0.25% PVP-I.

16. The composition of claim 14 wherein the composition comprises 44% DMSO.

17. The composition of claim 14 wherein the composition further comprises a co-solvent.

18. The composition of claim 14 wherein the composition comprises a gelling agent in an amount effective for forming a stable gel composition.

19. The composition of claim 18 wherein the gelling agent is selected from gum, agar, carrageenan, petrolatum, and a cellulosic polymer.

20. The composition of claim 18 Wherein the gelling agent is a cellulosic polymer.

21. The composition of claim 20 wherein the cellulosic polymer is hydroxyethylcellulose.

22. The composition of claim 14 wherein the composition is administered to a non-human mammal.

23. The composition of claim 14 wherein the composition is administered to a feline and the herpesvirus is feline herpesvirus (FHV).

24. The composition of claim 14 wherein the composition is administered to a canine and the herpesvirus is canine herpesvirus (CHV).

25. The composition of claim 14 wherein the composition is administered to an equine and the herpesvirus is equine herpesvirus (EHV).

26. The composition of claim 14 wherein the composition is used for herpes keratitis.
